(19) United States
(12) Patent Application Publication
(10) Pub. No.: US 2005/0049210 A1
(43) Pub. Date: Mar. 3, 2005

(54) Methods for the controlled delivery of pharmacologically active compounds

(75) Inventors: Yerramilli V.S.N. Murthy, Apex, NC (US); Robert H. Suva, Windham, ME (US)

Correspondence Address: MORGAN LEWIS & BOCKIUS LLP, 1111 PENNSYLVANIA AVENUE NW, WASHINGTON, DC, 20004, US

(73) Assignee: IDEXX Laboratories, Inc.

(21) Appl. No.: 10/650,262

(22) Filed: Aug. 27, 2003

(51) Int. Cl. A61K 31/704; A61K 31/65; A61K 31/655; A61K 9/127

(52) U.S. Cl. 514/35; 514/152; 424/450; 514/156

(57) ABSTRACT

The present invention provides compositions and methods for extending the release times and lowering the toxicity of pharmacologically active compounds. The compounds comprise a salt of the pharmacologically active compound with a lipophilic counterion and a pharmaceutically acceptable water immiscible solvent. In one embodiment the compositions are provided as injectable compositions. The lipophilic counterion may be a saturated or unsaturated C₈-C₂₂ fatty acid, and preferably may be a saturated or unsaturated C₁₀-C₈ fatty acid. The compositions are released over time when administered to a mammal. Therefore, the present invention enables one to provide a controlled dose administration of the active compound for periods of up to 15 days or even longer. Many compounds can be administered according to the present invention including, but not limited to, tilmicosin, oxytetracycline, fluoxetine, roxithromycin, and turbinafine.

### FIELD OF THE INVENTION

The present invention relates to methods and compositions for extending the release times and decreasing the toxicity of pharmacologically active compounds.

### BACKGROUND OF THE INVENTION

The following discussion of the background of the invention is merely provided to aid the reader in understanding the invention and is not admitted to describe or constitute prior art to the present invention.

It is often desirable to extend the release time of an injected drug to increase its duration of action, or to reduce its toxic effects. Formulations that are readily soluble in the body are usually absorbed rapidly and provide a sudden burst of available drug as opposed to a more desirable and gradual release of the pharmacologically active product. A variety of attempts have been made to provide controlled and extended release pharmaceutical compounds, but have not succeeded in overcoming all of the problems associated the technology, such as achieving an extended release time, maximum stability and efficacy, reduced toxicity, maximum reproducibility in preparation, and the elimination of unwanted physical, biochemical, or toxicological effects introduced by undesirable matrix materials. Furthermore, the animal patient is generally unwilling to consume dosage administrations. Therefore, compositions and methods that would result in fewer administrations would lessen stress on the treated animal and provide for an easier and more secure dosage regimen for animal caretakers.

Oxytetracycline is a widely used and useful antibiotic for treating various infections in mammals. In particular it is used for treating and preventing respiratory infections in domestic animals. But significant costs are associated with repeated administrations through conventional means.

Tilmicosin is a macrolide antibiotic with two tertiary amines. It has a long tissue half-life and is effective against a broad range of bacteria and is used to treat respiratory diseases in cattle. At elevated levels tilmicosin is cardiotoxic. Therefore, its use in sensitive species such as cats, goats, pigs and horses has been avoided almost entirely due to safety reasons. The commercial product, MICOTIL® (Eli Lilly & Co., Indianapolis, Ind.), is a solution of the di-phosphate salt of tilmicosin and is described in U.S. Pat. No. 5,574,020. This formulation is effective in cattle, but the antibiotic is released rapidly and results in toxicity in many species, especially in smaller animals such as dogs and cats.

Fluoxetine (PROZAC®, Eli Lilly, Indianapolis, Ind.) is an antidepressant, antiobsessional, and antibulimic drug. The beneficial actions of fluoxetine are presumed to be linked to its ability to inhibit the neuronal reuptake of serotonin. Fluoxetine preferentially inhibits the reuptake of serotonin into brain synaptosomes and platelets. In addition to these effects in humans, Fluoxetine is also useful for the control of separation anxiety and overt aggression in dogs, and the control of urine spraying behavior in cats.

Terbinafine (LAMISIL®, Novartis, Basel, Switzerland) is a synthetic anti-fungal agent. Structurally, it is an allylamine related to naftifine. It is highly lipophilic in nature and tends to accumulate in skin, nails, and fatty tissues. Terbinafine is mainly effective against the group of fungi termed dermatophytes, and is therefore useful in the treatment of infections of the keratinized epithelium, such as ringworm in cats.

Any of the drugs described above, as well as many others, would find safer and more convenient use if available in a time release form.

### SUMMARY OF THE INVENTION

In one aspect the present invention provides compositions for the administration of a pharmacologically active compound to a mammal. The compositions contain a salt of a pharmacologically active compound with a lipophilic counterion, and a pharmaceutically acceptable, water immiscible solvent. The ingredients are combined together to form an injectable composition that releases the active compound over time when administered to the mammal. In one embodiment the composition is injected into the mammal. The pharmacologically active compound can be an antibiotic. In various embodiments the pharmacologically active compound is tilmicosin, fluoxetine, oxytetracycline, doxycycline, roxithromycin, azithromycin, terbinafine, trimethoprim, neomycin, streptomycin, gentamycin, dibucaine, bupivacaine, benzocaine, tetracaine, acepromazine, itraconazole, tetracyclines, sulfonamides, or an aminoglycoside. In some embodiments the lipophilic counterion is an ionized form of a C₁₀-C₂₂ saturated or un-saturated fatty acid, while in other embodiments the lipophilic counterion is an ionized form of a C₁₀-C₈ saturated or unsaturated fatty acid. Examples of suitable fatty acids include, but are not limited to, lauric acid, decanoic acid, myristic acid, oleic acid and linoleic acid, or combinations thereof. In one embodiment the composition is a clear solution.

In another embodiment the lipophilic counterion is an ionized form of a polycarboxylic acid such as, for example, sebacic acid, polysebacic acid, polyaspartic acid, polyacrylic acid, or polybenzoic acid, or combinations thereof. In various embodiments the pharmaceutically acceptable water immiscible solvent is saw flower oil, safflower oil, castor oil, isopropyl myristate, soybean oil, cottonseed oil, corn oil, sunflower oil, arachis oil, olive oil, a medium or long chain fatty acid, ethyl oleate, linoleic acid, isopropyl palmitate, a glycerol ester, a polyoxyl hydrogenated castor oil, cod liver oil, a fish derived oil, coconut oil, or combinations thereof. Other water immiscible solvents can also be identified that will find use in the present invention. In one embodiment the mixture of active compound and water immiscible solvent forms a clear solution at room temperature.

In one embodiment of the invention the pharmacologically active compound is tilmicosin, the lipophilic counterion is an ionized form of linoleic acid, and the pharmaceutically acceptable solvent is one or more of saw flower oil, safflower oil, castor oil, and isopropyl myristate. In yet another embodiment the pharmacologically active compound is fluoxetine, the lipophilic counterion is an ionized form of decanoic acid, and the pharmaceutically acceptable solvent is one or a combination of safflower oil, castor oil, and isopropyl myristate. In another embodiment the compositions of the invention form a biphasic mixture when injected into water. By “biphasic mixture” is meant that two phases are formed having distinct partition coefficients. Thus, the compositions are not soluble in water but rather form a cohesive oily mass when injected into water. The cohesive oily mass maintains a distinct phase separation from the aqueous phase.

By “salt” is meant two compounds that are not covalently linked but are chemically bound through ionic attractions. The bond in a salt may be the result of a combination of an ionic bond and a hydrogen bond. Thus, for example, a “linoleic acid salt” of tilmicosin refers to tilmicosin bound to an ionized form of linoleic acid through an ionic attraction. In one embodiment the formulations of the present invention are non-aqueous. By “water immiscible” is meant that the solvent is incapable of mixing in a 1% or greater ratio in water without separation of two phases at 98° F. By “polycarboxylic acid” is meant a molecule containing at least two carboxyl groups. In various embodiments the polycarboxylic acid is polyaspartic acid, polyacrylic acid, sebacic acid, dodecanedioic acid, polysebacic acid, polybenzoic acid, or combinations thereof. By “poly” is meant two or more. As used herein, “about” refers to the range including the indicated value plus or minus 10%. By a “clear solution” is meant that the solution can be passed through a filter and no precipitate is collected on the filter. In one embodiment the filter has pore sizes of 0.22 um, and in another embodiment the filter has pore sizes of 0.45 um. Clear solutions are not suspensions. Clear solutions can be obtained by filtration through a 0.22 um filter or a 0.45 um filter.

By a “lipophilic counterion” is meant an ionized form of a fat soluble molecule. The lipophilic counterion may be an ionized form of a fatty acid, but may also be another fat soluble molecule. The counterion has at least one charge opposite to that of a chemical group on an opposing salt member, thereby causing an ionic attraction between the two molecules. The particular water/octanol partition coefficient of a lipophilic counterion will vary. In one embodiment the lipophilic counterions have a water/octanol partition coefficient of 100 or greater. In other embodiments the coefficient is 50 or greater (e.g., benzoic acid), or 40 or greater, or 25 or greater, or 10 or greater.

In another aspect the present invention provides a composition of a linoleic acid salt of tilmicosin and a pharmaceutically acceptable water immiscible solvent. At least a portion of the linoleic acid salt of tilmicosin is dissolved in the solvent. In one embodiment the linoleic acid salt of tilmicosin dissolves completely in the solvent and forms a clear solution. The pharmaceutically acceptable solvent is safflower oil, castor oil, or isopropyl myristate, or a combination thereof In one embodiment the linoleic acid is present at about 2 molar equivalents to the tilmicosin, and in other embodiments is present at from about 1.5 molar equivalents to about 2.5 molar equivalents or more. By “di(linoleic)” acid salt is meant two linoleic acid molecules bound to the tilmicosin. By “at least a portion” is meant at least 10%. In various other embodiments “at least a portion” means at least 25% or 35% or 50% or 65% or 75% or 90% of the salt dissolves in the solvent and forms a biphasic solution in water. A salt “dissolves” in the solvent when no more than 10% of the salt is retained on a 0.22 um filter when the sample is mixed with the solvent and filtered at 98° F.±2° F.

In another aspect the present invention provides methods for administering a pharmacologically active compound to a mammal. The methods involve administering to the mammal a composition of the invention described herein. In one embodiment the composition is administered by injecting it into the mammal. In various embodiments the pharmacologically active compound is an antibiotic such as, for example, tilmicosin, terbinafine, trimethoprim, neomycin, streptomycin, gentamycin, tetracyclines, sulfonamides, tilmicosin, and aminoglycosides. In another embodiment the pharmacologically active compound is fluoxetine. The methods involve administering active compounds that, if presented in previously available forms, may result in toxicity to the treated mammal. Thus, the formulations and methods of the present invention enable one to administer compounds that previously have not been able to be widely used in particular mammals due to safety considerations. In other embodiments the composition can be administered orally, sub-cutaneously, intra-peritoneally, intra-dermally, intra-muscularly, mucosally, or by other means.

By releasing the pharmacologically active compound “over time” is meant that the active compound is present in the blood or treated tissue of the mammal at a pharmaceutically effective amount for at least 2 days after administration. In other embodiments the active compound is present in the blood or treated tissue of the mammal for at least 3 days, at least 4 days, or at least 5 days, or at least 6 days, or at least 7 days, or at least 8 days, at least 9 days, or at least 10 days, or at least 11 days, at least 12 days, or at least 13 days, or at least 14 days, or at least 15 days, or at least 20 days, or at least 25 days, or at least 30 days, or at least 35 days, or at least 40 days, or at least 45 days, or at least 50 days, or at least 55 days, or at least 60 days. The methods therefore enable one to extend the release times of compounds and provide a controlled dose of active compound to the treated patient. The precise time will depend on several variables that may be manipulated to optimize the present invention for a particular pharmacologically active compound or application. In various embodiments the compound is present in the blood or treated tissue at a pharmaceutically effective amount for the time periods described above after injection. In another embodiment a pharmaceutically effective amount is present in the treated tissue for the number of days indicated after a single injection. The “treated tissue” is the tissue sought to be treated, for example lung tissue or other tissue to which the active compound is to be delivered. By “pharmaceutically effective amount” is meant an amount that exerts a measurable and medically significant effect on the treated mammal, resulting in progress towards curing, arresting, or preventing the subject disease, or alleviating or preventing the condition that was the reason for treatment. Some drugs (e.g., tilmicosin) accumulate in the tissues and are present in a lower amount in the blood serum. Thus, an amount may be present in the blood that does not have a measurable and medically significant effect, but the amount of drug in the blood may correlate with a higher concentration accumulated in target tissues, and therefore constitutes a pharmaceutically effective amount. A “pharmaceutically acceptable solvent” is a liquid that dissolves a salt of the pharmacologically active compound and a lipophilic counterion, and that is suitable for use with humans and/or animals without undue adverse side effects (such as toxicity, irritation, and allergic response), and commensurate with a reasonable benefit/risk ratio.

In another aspect the present invention provides methods of manufacturing a formulation of the invention. The methods involve producing a composition of the present invention by producing a salt of a pharmacologically active compound with a lipophilic counterion in a pharmaceutically acceptable water immiscible solvent to manufacture a formulation of the invention. The formulation releases the active compound over time when administered to the mammal as described herein. In one embodiment the formulation is an injectable formulation. In other embodiments the formulation can be administered orally, subcutaneously, intre-peritoneally, intra-dermally, mucosally, or by other methods.

In another aspect, the present invention provides methods of extending the release time and lowering the toxicity of a pharmacologically active compound administered to a mammal. The methods comprise preparing a formulation of the invention, and administering the composition to the mammal as described herein. The composition releases the pharmacologically active compound over time after administration to the mammal, thereby extending the release time of the compound. The invention may therefore provide a controlled dosage of active compound to the treated mammal. The present invention enables one to provide a controlled dose administration of the active compound for periods of at least 2-15 days, or at least 20 days, or at least 30 days, or at least 40 days, or at least 50 days, or at least 60 days, or even longer, as described above.

In another aspect the present invention provides compositions for the administration of a pharmacologically active compound to a mammal. The compositions contain a salt of the pharmacologically active compound with a lipophilic counterion and a water immiscible pharmaceutically acceptable solvent, combined together to form an injectable composition described herein. The present invention therefore offers important advantages over formulations previously available. The present invention allows for the controlled release of pharmacologically active compounds resulting in reduced toxicity, particularly in small animals such as dogs and cats. It also offers the advantage of being able to administer compounds to domestic animals in an efficient manner, thereby requiring a smaller investment in time and resources than is available with previous modes of drug administration. The pharmacologically active compound is available in a stable, injectable, formulation that slowly releases the active compound over an extended period of time after injection into a mammal. By “injectable formulation” or “injectable composition” is meant a formulation or composition that can be injected, i.e., drawn into a syringe and injected subcutaneously, intraperitoneally, or intra-muscularly into a mammal without causing adverse effects due to the presence of solid materials in the composition. Solid materials include, but are not limited to, particles, crystals, a gummy mass, and a gel. By “pharmacologically active compound” is meant a chemical compound that causes a pharmacological effect in the treated mammal. For example, the effect may be to destroy, hinder, or prevent growth of bacteria or parasites, reduce inflammation, calm, tranquilize, or decrease aggression in the mammal, or another pharmaceutical and measurable effect in the treated mammal.

In one embodiment the composition of the invention has a tilmicosin concentration of from about 100 mg/ml to about 600 mg/ml and is injected at a dose of from about 10 mg tilmicosin/kg to about 45 mg/tilmicosin/kg of mammal. The composition can have a tilmicosin concentration of about 300 mg/ml. In various embodiments, the composition has a tilmicosin concentration of about 200 mg/ml, about 300 mg/ml, about 400 mg/ml, or about 500 mg/ml. In still other embodiments the dose is about 20 mg/kg, about 30 mg/kg, or about 40 mg/kg. The mammal may be a dog or a cat, such as the common house cat Felis catus.

In another aspect the invention provides methods for treating mammals in need of such treatment. The methods involve administering to a mammal a composition of the invention according to the methods described herein. The methods can be used to treat various diseases and conditions, such as Lyme disease, pustular contagious dermatitis, soft tissue infections, an ear infection, and a urinary tract infection. Lyme disease occurs in humans, dogs, cats, horses, and cattle, and is caused by the bacterial spirochete Borrelia (e.g., Borrelia burgdorferi). Pustular contagious dermatitis (also called “orf”) is a cutaneous lesion caused by a parapox virus that occurs in sheep. Clinical features include one or more red lesions that develop on the udder and teats of the sheep. Soft tissue infections are a severe type of tissue infection that can involve the skin, subcutaneous fat, the muscle sheath (fascia), and the muscle. These infections can cause gangrenous changes, tissue death, systemic disease, and frequently death. Soft tissue infections are most often caused by Staphylococcus aureus (Gram-positive cocci in clusters) and Streptococcus pyogenes (Gram-positive cocci in chains). The present invention is also applicable to the treatment of other types of infections, and those described herein are provided by way of specific examples.

In another aspect the present invention provides methods of treating respiratory disease in a mammal. The methods involve administering to the mammal a composition of the present invention according to the methods described herein. In various embodiments the respiratory disease is caused by the organism Bordetella bronchiseptica. This organism is a Gram-negative, aerobic coccobacillus and is a primary pathogen responsible for respiratory disease in many animal species. The organism inhabits the upper respiratory tract, but is associated with both upper and lower airway disease. The organism is responsible for causing various respiratory diseases. For example, in dogs the organism causes kennel cough (tracheobronchitis), rhinitis, and sinitus. In cats the organism causes tracheitis, suppurative bronchopneumonia, and lymphadenitis. In pigs the organism causes atrophic rhinitis and pneumonia in pigs, and also causes respiratory diseases in rabbits such as “snuffles” and serous to purulent rhinitis. In guinea pigs the organism causes serous to purulent otitis media, necrotizing tracheitis, suppurative necrotizing bronchopneumonia. In rats the organism causes acute to subacute bronchopneumonia and atrophic rhinitis, as well as causing respiratory diseases in primates. The organism is transferred by direct animal to animal contact, as well as by airborne transmission. The terms of diseases described above are intended according to their standard clinical definitions.

In other embodiments the compositions and methods of the invention are useful for treating respiratory diseases in mammals associated with mycoplasma. For example, mycoplasma hypopneumoniae is an obligatory pathogen of the respiratory tracts of pigs and rodents. The organisms infects the airways of the respiratory system and is recognized as an initiator or potentiator of more serious respiratory diseases caused by other primary or opportunistic pathogens. By “associated with” mycoplasma means that the presence of mycoplasma is an initiator or potentiator of the disease.

The summary of the invention described above is not limiting and other features and advantages of the invention will be apparent from the following detailed description of the preferred embodiments, as well as from the claims.

### BRIEF DESCRIPTION OF THE DRAWINGS

FIG. 1 is a graphical illustration showing the in vitro release kinetics of a tilicosin-linoleic acid fatty acid salt of the present invention in various water immiscible solvents. The illustration shows that tilmicosin prepared according to the present invention is released into saline at a rate slower than that of the free tilmicosin (MICOTIL®, Elanco, Indianapolis, Ind.). The solvents are safflower oil, castor oil, and isopropyl myristate.

FIG. 2 is a graphical illustration showing the pharmacokinetics of tilmicosin-linoleic fatty acid salt (FAS) in linoleic acid as the water immiscible solvent in dog and cat serum.

FIG. 3 is a graphical depiction illustrating the pharmacokinetics of tilmicosin fatty acid salt (FAS) in isopropyl myristate as the water immiscible solvent in dog serum.

### DETAILED DESCRIPTION OF THE INVENTION

The compositions of the present invention may be prepared using salts of pharmacologically active compounds with basic functionalities. These can be made using the ionized forms of a variety of lipophilic acids, saturated or unsaturated fatty acids, cholic acids, phosphatidic acids, dicarboxylic acids such as sebacic acid or any acid that, when combined with the pharmacologically active compound, renders the resulting salt insoluble in water, but soluble in a water immiscible solvent. By “water insoluble” is meant that the solvent does not have a significant level of solubility in aqueous solutions.

The compositions of the present invention offer several advantages. The compositions contain high concentrations of the active compound. In various embodiments, the pharmacologically active compound may be contained in the composition in the range of 10%-60% (w/v). But this range may be varied widely, depending on the solubility or insolubility of the pharmacologically active compound, the lipophilic counterion selected, the water immiscible solvent selected, the injectability (or non-injectability) of the final product, and any other relevant needs of the particular application. For example, pharmacologically active compound can be contained in the composition at a concentration of at least 20%, at least 30%, at least 40%, or at least 50% (w/v), and may also be present as low as 10%, or 5%, or even 1% and still provide a useful effect. Similarly, active compound may be present at about 60%, about 70%, about 80%, about 90%, or even higher as needs require. No exotic excipients or carriers are required. The compositions are easily filtered, thereby simplifying the manufacturing process. It is believed that the exclusion of water from the formulation should impart greater stability to the formulation, and inhibit the growth of microorganisms. The processes for preparing the compositions, as described herein, are simple. When injected, administration of the compositions according to the present invention results in milder reactions at the injection site due to the neutralization of the pharmacologically active compound.

Another advantage of the present compositions is that they are delivered in a water immiscible solvent. Thus, the solvent does not diffuse off into the body, but rather exists for a period of time as a cohesive oily mass within the body, and therefore acts as a drug depot. The water immiscible solvents are lipophilic and therefore can solubilize lipophilic drugs at high concentrations. Thus, pharmacologically active substances that are not soluble in water can be delivered according to the present invention. For example, the anti-fungal agent terbinafine is such a substance.

The present invention provides the ability to modulate the release rate and release time of the pharmacologically active compound. The release rate may be modulated by varying the lipophilicity and molecular weight of the counter-ion used to make the salt. For example, linoleic acid salts of tilmicosin are usually released more slowly than decanoate salts. In addition, higher concentrations of the salt in the formulation usually yield slower release rates. For example, the decanoate salt of tilmicosin is released more slowly from a 60% tilmicosin-fatty acid salt formulation than from a 30% tilmicosin-fatty acid salt formulation. Similarly, as explained herein, other variables such as selection of lipophilic counterion, solvent selection, salt concentration, and others may be manipulated to lengthen or shorten the release time of the active compound to the desired point. Generally, it may be desirable for salts to be based on the molar ratio of charged groups. But one may also successfully create salts by utilizing a hemi-salt or by otherwise varying from a 1:1 ratio. The pharmaceutically acceptable solvent may be a water immiscible or water insoluble solvent. Mixtures of water insoluble and/or water immiscible solvents may also be utilized. Various water insoluble solvents may be mixed to optimize the result for a particular application. For example, a mixture of safflower oil, castor oil, isopropyl myristate, or other water immiscible solvents may be mixed in various ratios to provide an optimal solvent. In some embodiments, mixing in approximately even amounts may provide a suitable solvent.

In other embodiments, formulations of the invention containing a salt of the pharmacologically active compound with a lipophilic counterion can be combined with the unsalted form of the active, in order to provide a greater initial dose of active compound.

Without wanting to be bound by any particular theory, injectable compositions may be obtained when a salt is formed of a pharmacologically active agent with a lipophilic counterion, and combined with a parenteral organic solvent. It is believed that when this formulation is injected into a mammal, the formulation (both the salt of the pharmacologically active compound and the lipophilic counterion and the water immiscible solvent) form a drug depot in the mammalian body. In one embodiment the drug depot is formed under the skin containing the composition in its entirety. Over a period of time the pharmacologically active compound diffuses away from the drug depot and is released into the body. There will thus exist a concentration of the active compound that is released in a pharmaceutically effective amount over a desired period of time. Release times may be obtained of at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 20 days, at least 25 days, at least 30 days, at least 35 days, at least 40 days, at least 45 days, at least 50 days, at least 55 days, at least 60 days, or even longer, as desired. Since the compositions are not miscible in water, they are believed to exist in a distinct phase in the mammalian body that is distinct from body fluids such as blood. Thus, two phases are present.

By “drug depot” is meant a concentration or precipitation of pharmacologically active compound within the body of the treated mammal that releases a pharmaceutically effective amount of the active compound over time. In one embodiment the pharmacologically active compound is released over a period of 2 days or greater and is present in the blood or tissue of the mammal at a pharmaceutically effective amount for that period. In other embodiments, the compound is released and present at a pharmaceutically effective amount over a period of 3 days or 4 days or 5 days or greater as described above. The drug depot can be formed by any type of injection, where the composition forms a depot or pool in the mammalian body. A drug depot can also be formed with other modes of administration, such as by oral administration. In this embodiment the drug depot can be a concentration of cohesive oily mass present in the digestive tract of the mammal. The composition can also form a drug depot by being injected under the skin of the mammal.

In another embodiment the compositions are administered orally to the mammal. The compositions are present in the digestive tract of the mammal as a cohesive, oily mass and move slowly through the digestive tract. The compositions may also become extended over a distance within the digestive tract. The pharmacologically active compound is slowly absorbed through the gut and a time release of the compound is achieved.

Fatty acid chain length, the particular combinations of fatty acids, the percent pharmacologically active compound:lipophilic counterion salt in the formulation, and the pharmaceutically acceptable water immiscible solvent selected all influence the release kinetics of the pharmacologically active compound. With reference to the present disclosure, the release kinetics of the pharmacologically active compound may be conveniently and easily managed by manipulating these and other variables. It was also found that the formulations retained stability during sterilization by autoclave. The present invention may be applied to many pharmacologically active compounds that have an appropriate solubility and chemical functionality. Thus, it is contemplated that the present invention may be applicable to a wide variety of pharmacologically active compounds, such as drugs, medications, nutrients, or other desirable compounds for administration to a mammal.

Some modifications to the methods presented herein may be desirable based on the particular characteristics of the pharmacologically active compound involved. The following non-limiting examples present further applications of the present invention and are provided by way of example only.

### EXAMPLE 1

Oxytetracycline

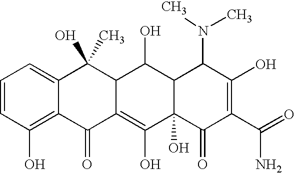

Oxytetracycline has one tertiary amine group, and the hydrochloride salt of oxytetracycline is readily soluble in water. Adding one mole of fatty acid to one mole of oxytetracycline creates a salt that is more soluble than unmodified oxytetracycline in isopropyl myristate.

An oxytetracycline composition according to the present invention was prepared by adding 0.464 grams of oxytetracycline and 0.203 grams of lauric acid to 3 ml of isopropyl myristate. The mixture is stirred for 60 minutes, resulting in a clear solution at 25° C.

### EXAMPLE 2

Tilmicosin

Tilmicosin is an antibiotic in the macrolide class with the following structure:

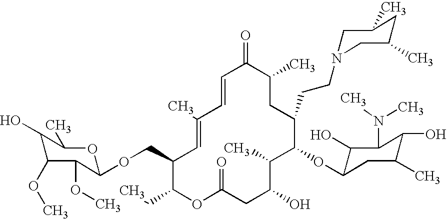

It is effective against a broad range of bacteria, and is used for the treatment of respiratory diseases in cattle. The basic form is moderately soluble in aqueous solvents, while the chloride and phosphate salts are highly soluble. At elevated levels, tilmicosin is cardiotoxic, and therefore is not administered intravenously. For safety reasons, its use has been avoided almost entirely in sensitive species such as cats, goats, pigs, and horses.

When the two amine groups of tilmicosin are neutralized with any of several fatty acids (such as, for example, decanoic C₁₀, lauric C₁₂, myristic C₁₄, palmitic C₁₆, stearic C₁₈, oleic C₁₈, elaidic C₁₈, linoleic C₁₈, and erucic C₂₂, sebacic, dodecanedioic), the resulting salt is soluble in pharmaceutically-acceptable water immiscible solvents. When a formulation of the salt (tilmicosin-linoleic acid) is sealed in a dialysis cassette and place in saline, the tilmicosin salt remains a cohesive mass, and tilmicosin is slowly released from the bag. The rate of release is a function of the chain length of the fatty acid, the solvent, and the tilmicosin-fatty acid salt concentration.

### EXAMPLE 3

Roxithromycin:

Roxithromycin is an antibiotic in the macrolide class with the following structure:

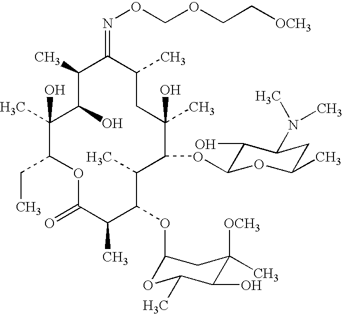

Roxithromycin is effective against a broad range of bacteria, and is used for the treatment of respiratory diseases in cattle. The amine group of roxithromycin can be neutralized with linoleic acid in isopropyl myristate, and a clear solution is obtained at 200 mg/ml.

### EXAMPLE 4

Terbinafine

Terbinafine is an antifungal and the structure is as shown below:

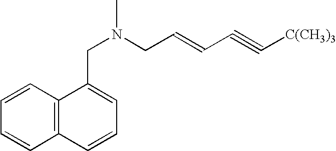

Terbinafine is a specific inhibitor of squaline epoxidase, a key enzyme in fungal ergosterol biosynthesis. The amine group of terbinafine was neutralized with linoleic acid in ispropyl myristate, resulting in a clear solution at 150 mg/ml. The resulting salt was highly insoluble in water.

### EXAMPLE 5

Azithromycin

Azithromycin is an antibiotic and belongs to the macrolide class with the following structure:

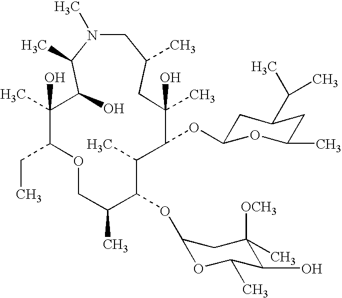

The basic form is moderately soluble in aqueous solvents, while the chloride and phosphate salts are highly soluble. When the two basic amine groups of azithromycin were neutralized with hydrophobic fatty acids, it resulted in a salt that was soluble in pharmaceutically-acceptable water immiscible solvents.

Preparation of a typical 10% azithromycin-linoleic acid fatty acid salt formulation in water immiscible solvent was achieved as follows: Five grams of azithromycin and 3.4 grams of linoleic acid were weighed into a 50 ml volumetric flask and the volume adjusted to 90% with isopropyl myristate. The flask was placed on a shaker and agitated until a clear solution was obtained. The volume was adjusted to 50 ml with isopropyl myristate.

### EXAMPLE 6

Preparation of Tilmicosin-Fatty Acid Salts

Hydrophobic water insoluble tilmicosin-fatty acid salts were formulated in water immiscible solvents such as castor oil safflower oil, and isopropyl myristate. Formulations were also prepared using the fatty acid linoleic acid itself as the pharmaceutically acceptable water immiscible solvent.

Preparation of a typical 10% tilmicosin-linoleic acid FAS formulation in water immiscible solvent was achieved as follows: Five grams of tilmicosin and 3.4 grams of linoleic acid were placed in a 50 ml volumetric flask and the volume adjusted to 50 ml with the appropriate solvent (e.g., castor oil, safflower oil, or isopropyl myristate). The flask was placed on a shaker and agitated until a clear solution was obtained.

The preparation of 20% tilmicosin-linoleic FAS with linoleic acid as the pharmaceutically acceptable water immiscible solvent was achieved as follows: Five grams of tilmicosin powder was placed into a 25 ml volumetric flask and the volume adjusted to 25 ml with linoleic acid. The flask was placed on a shaker until a clear solution was obtained.

### EXAMPLE 7

In Vitro Release Kinetics of Tilmicosin-Fatty Acid Salts

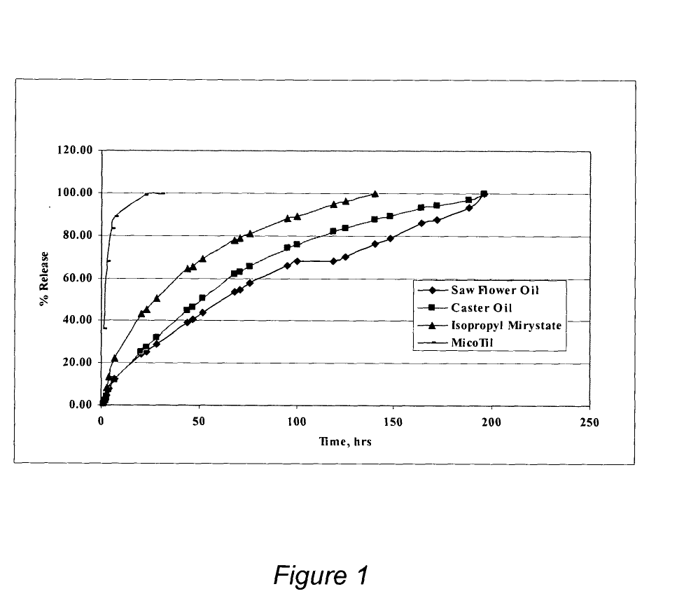

The in vitro release kinetics of tilmicosin-FAS in a water immiscible solvent was examined as follows: 0.5 ml of 10% tilmicosin-linoleic acid formulation in various water immiscible solvents including safflower oil, castor oil, and isopropyl myristate. The formulations were injected into different dialysis cassettes (Pierce) and the release kinetics were compared to that of unmodified MICOTIL® (Eli Lilly, Indianapolis, Ind.), which is a commercially available phosphate salt of tilmicosin. The cassettes were placed in different reservoirs of phosphate buffered saline (PBS) at 37° C. in an incubator shaker. One ml aliquots of PBS were removed from reservoirs at several time points and analyzed for tilmicosin using HPLC. The data is presented in FIG. 1. The results indicate that while the half-life for MICOTIL® is about 90 minutes, it is 40-55 hours for the tilmicosin formulations of the present inventions. In vitro release of active compound was observed for about a week using the compositions of the invention while the release is completed within a few hours for MICOTIL®.

### EXAMPLE 8

In Vivo Release Kinetics of a Tilmicosin-Linoleic Acid Salt in Linoleic Acid

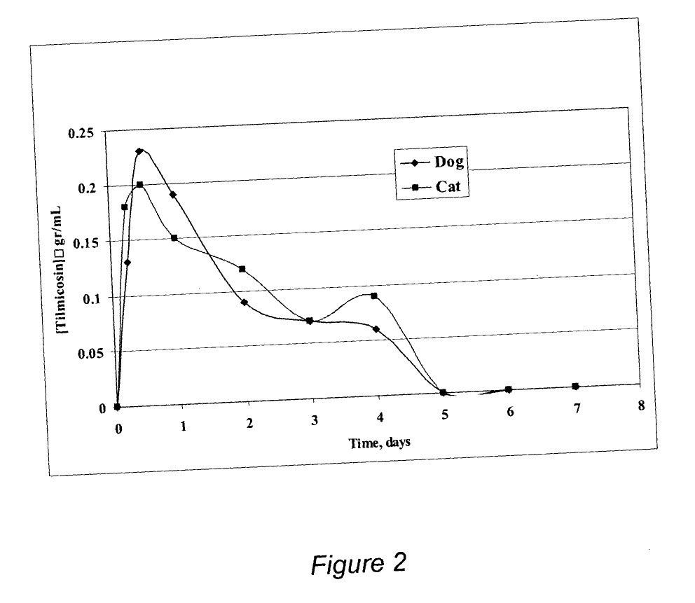

20% tilmicosin as linoleic acid salt was formulated with linoleic acid as the water immiscible solvent. The formulation was injected subcutaneously at 10 mg/kg into one dog and one cat. Serum samples were collected at several time points and analyzed for tilmicosin concentration using solid phase extraction and HPLC techniques. The data is illustrated in FIG. 2 and shows that tilmicosin was detected in the dog and cat serum for about 4 days, indicating a controlled release of the drug into the serum.

### EXAMPLE 9

In Vivo Release Kinetics of a Tilmicosin-Linoleic Acid Salt in Isopropyl Myristate

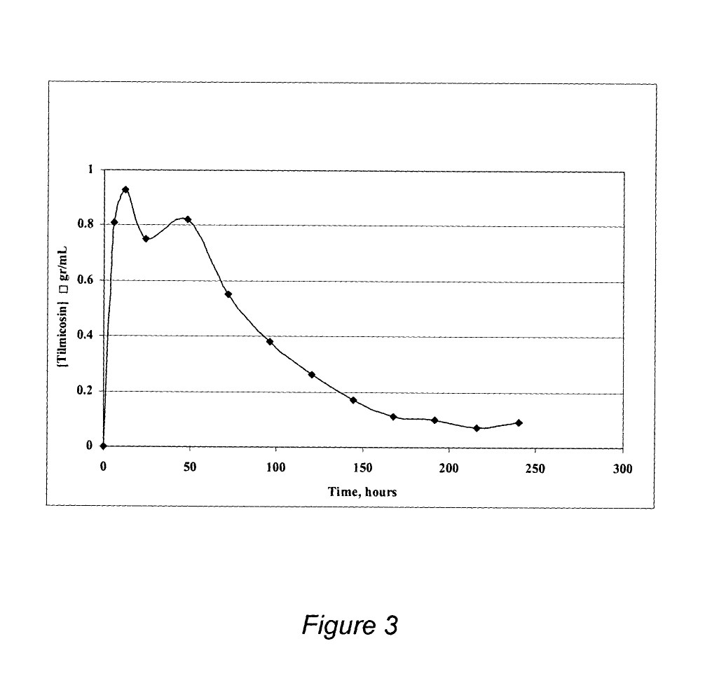

20% tilmicosin as linoleic acid salt was formulated in isopropyl myristate as the water immiscible solvent. The formulation was injected subcutaneously at 50 mg/kg into a dog. Serum samples were collected at several time points and analyzed for tilmicosin concentration using solid phase extraction and HPLC techniques. The data are presented in FIG. 3 and illustrate that tilmicosin was detected at pharmaceutically effective amounts in the dog serum for about 5 days.

A 50 mg/kg dose of tilmicosin is normally lethal in dogs, and these data therefore prove that tilmicosin can be safely administered to dogs according to the present invention and achieve a controlled release of tilmicosin. The 5 day presence in dog serum also indicates a controlled release of the drug into the serum.

### EXAMPLE 10

Preparation of Fluoxetine-FAS Formulations

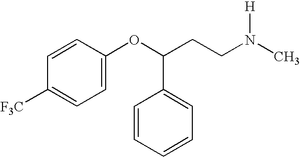

Fluoxetine (PROZAC®, Eli Lilly, Indianapolis, Ind.) is a selective serotonin reuptake inhibitor and is extensively used to treat psychological disorders such as obsessive-compulsive disorder in humans. It is also effective for treating aggressive behavior and separation anxiety in dogs and urine spraying behavior in cats.

10% Fluoxetine-FAS was formulated in different water immiscible solvents as described below.

2.5 g of fluoxetine and 1.529 g of decanoic acid were placed into a 25 ml volumetric flask and the volume adjusted to 25 ml with isopropyl myristate. The flask was placed on a shaker and agitated until a clear homogeneous solution was obtained.

2.5 g of fluoxetine and 2.512 g of oleic acid were placed into a 25 ml volumetric flask and the volume adjusted to 25 ml with safflower oil. The flask was placed on a shaker and agitated until a clear homogeneous solution was obtained.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

With reference to the present disclosure, it will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are set forth within the following claims.

1. A composition for the administration of a pharmacologically active compound to a mammal, comprising:
a salt of the pharmacologically active compound with a lipophilic counterion; and
a pharmaceutically acceptable, water immiscible solvent;
combined together to form a composition that releases the active compound over time when administered to the mammal.

2. The composition of claim 1 wherein the composition is an injectable composition.

3. The composition of claim 2 wherein the pharmacologically active compound is an antibiotic.

4. The composition of claim 2 wherein the pharmacologically active compound is selected from the group consisting of: tilmicosin, fluoxetine, oxytetracycline, doxycycline, roxithromycin, terbinafine, trimethoprim, neomycin, streptomycin, gentamycin, dibucaine, bupivacaine, benzocaine, tetracaine, acepromazine, itraconazole, tetracyclines, sulfonamides, and aminoglycosides.

5. The composition of claim 4 wherein the pharmacologically active compound is tilmicosin, terbinafine, or fluoxetine.

6. The composition of claim 2 wherein the lipophilic counterion is a C₁₀-C₂₂ saturated or un-saturated fatty acid.

7. The composition of claim 2 wherein the lipophilic counterion is an ionized form of a C₁₀-C18 saturated or unsaturated fatty acid.

8. The composition of claim 7 wherein the fatty acid selected from the group consisting of one or more of: lauric acid, decanoic acid, myristic acid, oleic acid and linoleic acid.

9. The composition of claim 2 wherein the lipophilic counterion is an ionized form of a polycarboxylic acid.

10. The composition of claim 9 wherein the polycarboxylic acid is selected from the group consisting of one or more of: sebacic acid, polysebacic acid, polyaspartic acid, polyacrylic acid, and polybenzoic acid.

11. The composition of claim 2 wherein the pharmaceutically acceptable water immiscible solvent is selected from the group consisting of one or more of: saw flower oil, safflower oil, castor oil, isopropyl myristate, ethyl lactate, soybean oil, cottonseed oil, corn oil, sunflower oil, arachis oil, olive oil, palm oil, coconut oil, hemp seed oil, canola oil, almond oil, glycerin, a medium or long chain fatty acid, ethyl oleate, linoleic acid, isopropyl palmitate, a glycerol ester, a polyoxyl hydrogenated castor oil, cod liver oil, and a fish derived oil.

12. The composition of claim 11 wherein the pharmaceutically acceptable water immiscible solvent is selected from the group consisting of one or more of: safflower oil, castor oil, linoleic acid, and isopropyl myristate.

13. The composition of claim 2 wherein the pharmacologically active compound is tilmicosin, the lipophilic counterion is linoleic acid, and the pharmaceutically acceptable solvent is selected from the group consisting of one or more of safflower oil, castor oil, and isopropyl myristate.

14. The composition of claim 2 wherein the pharmacologically active compound is fluoxetine, the lipophilic counterion is decanoic acid, and the pharmaceutically acceptable solvent is selected from the group consisting of one or more of: safflower oil, castor oil, and isopropyl myristate.

15. A method of administering a pharmacologically active compound to a mammal comprising:
administering to the mammal a composition comprising
a salt of the pharmacologically active compound and a lipophilic counterion in a pharmaceutically acceptable water immiscible solvent;
wherein the composition releases the active compound over time when administered to the mammal.

16. The method of claim 15 wherein the composition is administered to the mammal by injection.

17. The method of claim 16 wherein the pharmacologically active compound is an antibiotic.

18. The method of claim 17 wherein the antibiotic is selected from the group consisting of: tilmicosin, terbinafine, trimethoprim, neomycin, streptomycin, gentamycin, tetracyclines, sulfonamides, and aminoglycosides.

19. The method of claim 16 wherein the pharmacologically active compound is selected from the group consisting of: tilmicosin, terbinafine, and fluoxetine.

20. The method of claim 16 wherein the lipophilic counterion is an ionized form of a fatty acid.

21. The method of claim 20 wherein the fatty acid is an ionized form of a C₁₀-C₂₂ fatty acid.

22. The method of claim 21 wherein the fatty acid is selected from the group consisting of one or more of: lauric acid, decanoic acid, myristic acid, oleic acid and linoleic acid.

23. The method of claim 16 wherein the lipophilic counterion is an ionized form of sebacic acid.

24. The method of claim 16 wherein the pharmaceutically acceptable water immiscible solvent is selected from the group consisting of one or a combination of: saw flower oil, safflower oil, castor oil, isopropyl myristate, ethyl lactate, soybean oil, cottonseed oil, corn oil, sunflower oil, arachis oil, olive oil, a medium or long chain fatty acid, ethyl oleate, linoleic acid, isopropyl palmitate, a glycerol ester, a polyoxyl hydrogenated castor oil, cod liver oil, a fish derived oil, and coconut oil.

25. The method of claim 24 wherein the pharmaceutically acceptable water immiscible solvent is selected from the group consisting of one or more of: safflower oil, isopropyl myristate, and castor oil.

26. The method of claim 15 wherein the pharmacologically active compound is tilmicosin, the lipophilic counterion is linoleic acid, and the pharmaceutically acceptable solvent is selected from the group consisting of one or more of: safflower oil, castor oil, and isopropyl myristate.

27. The method of claim 15 wherein pharmacologically active compound is fluoxetine, the lipophilic counterion is decanoic acid, and the pharmaceutically acceptable solvent is selected from the group consisting of one or more: of safflower oil, isopropyl myristate, and castor oil.

28. The method of claim 15 wherein the active compound is present in the blood of the mammal at a pharmaceutically effective amount for at least 3 days.

29. The method of claim 15 wherein the active compound is present in the blood of the mammal at a pharmaceutically effective amount for at least 5 days.

30. The method of claim 15 wherein the composition is administered to the mammal orally.

31. A composition for administration of a pharmacologically active compound to a mammal, comprising:
a salt of the pharmacologically active compound with a lipophilic counterion; and
a pharmaceutically acceptable water immiscible solvent;
combined together to form a composition that forms a biphasic mixture when injected into water.

32. The composition of claim 31 wherein the pharmacologically active compound is an antibiotic.

33. The composition of claim 31 wherein the pharmacologically active compound is selected from the group consisting of: tilmicosin, terbinafine, fluoxetine, oxytetracycline, doxycycline, roxithromycin, terbinafine, trimethoprim, neomycin, streptomycin, gentamycin, dibucaine, bupivacaine, benzocaine, tetracaine, acepromazine, itraconazole, tetracyclines, sulfonamides, and aminoglycosides.

34. The composition of claim 33 wherein the pharmacologically active compound is tilmicosin, terbinafine, or fluoxetine.

35. The composition of claim 31 wherein the lipophilic counterion is a C₁₀-C₂₂ saturated or un-saturated fatty acid.

36. The composition of claim 31 wherein the lipophilic counterion is a C₁₀-C₁₈ saturated or unsaturated fatty acid.

37. The composition of claim 36 wherein the fatty acid selected from the group consisting of one or more of: lauric acid, decanoic acid, myristic acid, oleic acid and linoleic acid.

38. The composition of claim 31 wherein the lipophilic counterion is an ionized form of a polycarboxylic acid.

39. The composition of claim 38 wherein the polycarboxylic acid is selected from the group consisting of one or more of: sebacic acid, polysebacic acid, polyaspartic acid, polyacrylic acid, and polybenzoic acid.

40. The composition of claim 31 wherein the pharmaceutically acceptable water immiscible solvent is selected from the group consisting of one or a combination of: saw flower oil, safflower oil, castor oil, isopropyl myristate, ethyl lactate, soybean oil, cottonseed oil, corn oil, sunflower oil, arachis oil, olive oil, a medium or long chain fatty acid, ethyl oleate, linoleic acid, isopropyl palmitate, a glycerol ester, a polyoxyl hydrogenated castor oil, cod liver oil, a fish derived oil, and coconut oil.

41. The composition of claim 40 wherein the pharmaceutically acceptable water immiscible solvent is selected from the group consisting of: safflower oil, castor oil, linoleic acid, and isopropyl myristate.

42. The composition of claim 31 wherein the pharmacologically active compound is tilmicosin, the lipophilic counterion is linoleic acid, and the pharmaceutically acceptable solvent is selected from the group consisting of one or more of safflower oil, castor oil, and isopropyl myristate.

43. The composition of claim 31 wherein the pharmacologically active compound is fluoxetine, the lipophilic counterion is decanoic acid, and the pharmaceutically acceptable solvent is selected from the group consisting of one or more of: safflower oil, castor oil, and isopropyl myristate.

44. A composition for administration of a pharmacologically active compound to a mammal, comprising
a salt of the pharmacologically active compound with a lipophilic counterion; and
a pharmaceutically acceptable water immiscible solvent, combined together to form a clear solution.
